(19) United States
(12) Patent Application Publication
(10) Pub. No.: US 2012/0115872 A1
(43) Pub. Date: May 10, 2012

(54) COMBINATION OF A SODIUM-PROTON EXCHANGER INHIBITOR AND OF A DIHYDRO-1,3,5-TRIAZINE AMINE DERIVATIVE

(75) Inventors: Didier Mesangeau, Combx la Ville (FR); Daniel Cravo, Sartrouville (FR); Xavier Leverve, La Terrasse (FR)

(73) Assignee: Merck Patent GMBH, Darmstadt (DE)

(21) Appl. No.: 13/383,907

(22) PCT Filed: Jun. 18, 2010
(86) PCT No.: PCT/EP10/03696

§ 371 Date: Jan. 13, 2012

(30) Foreign Application Priority Data
Jul. 17, 2009 (FR) 0954978

(51) Int. Cl. A61K 31/53; A61P 9/06; A61P 9/00; A61P 9/10

(52) U.S. Cl. 514/245

(57) ABSTRACT

The invention relates to a combination of a sodium-proton exchanger inhibitor and of a dihydro-1,3,5-triazine amine derivative which is useful for the treatment and/or prevention of pathologies induced by ischaemia and/or reperfusion, especially cardiac and renal complications.

### FIELD OF THE INVENTION

The invention relates to a combination of a sodium-proton exchanger inhibitor and a dihydro-1,3,5-triazine amine derivative which is useful for the treatment and/or prevention of pathologies induced by ischaemia and/or reperfusion, especially cardiac and renal complications.

### TECHNICAL BACKGROUND

Myocardial ischaemia is defined as an imbalance between oxygen demand and supply. This imbalance leads to a disturbance of cardiac function. In the vast majority of cases, myocardial ischaemia is caused by an insufficiency of coronary blood circulation to the heart muscle tissue, thus depriving the myocardial cells of their oxygen supply or drastically reducing this supply. Such ischaemia may be due to the obstruction of a vessel (thrombosis), a reduction of the inside diameter of an artery (stenosis) or a decrease in the coronary blood flow (hypoperfusion), such as in states of circulatory insufficiency associated with severe sepsis with endotoxaemic shock. In this respect, it should be noted that severe sepsis also leads to haemodynamic dysfunction with direct myocardial depression. At the present time, however, it is not clear what mechanism is predominant in the reduction of myocardial function, hypoperfusion or myocardial depression by circulating cytokines.

Infarctions are one of the major consequences of ischaemias. The term “infarction” describes a focus circumscribed by tissue necrosis. Thus, myocardial infarction leads to the destruction of part of the heart due to the death of heart muscle cells. Myocardial infarction is a very common event. By way of example, it is estimated that in France, about 180 000 to 200 000 people per year are affected by this disease, which is predominant in men. It occurs primarily in individuals presenting cardiovascular risk factors, such as tobacco consumption, obesity, diabetes, hyperlipidaemia or arterial hypertension. The extent of myocardial infarction is a determining factor for contractile functional recovery of the myocardium and for the long-term prognosis of the patients.

Acute myocardial infarction (AMI) is an absolute cardiological emergency that involves management by specialized medical and hospital services with an acute-phase treatment whose aim is to reperfuse the ischaemic heart muscle and to prevent and/or limit the possible complications associated with infarction that often lead to the death of the patients within the first hours or the first days.

Reperfusion is defined as the re-establishment of an adequate blood circulation in an ischaemic tissue, enabling a balance to be re-established between oxygen demand and supply. Reperfusion during complete interruption of the coronary blood flow is generally achieved by unblocking of the occluded artery.

Although reperfusion unquestionably protects the myocardial cells from cell death caused by persistence of the ischaemia, it is also accompanied by deleterious effects on contractile function (myocardial sideration), heart rhythm (occurrence of arrhythmia) and tissue perfusion (“no reflow”). Recent data even indicate that paradoxically reperfusion might also kill some of the reperfused cells (reperfusion necrosis).

During reperfusion of a myocardial infarction, medicaments belonging to different therapeutic classes, for instance platelet aggregation inhibitors, such as acetylsalicylic acid, beta blockers, converting enzyme inhibitors (CEI) or statins, have a beneficial effect on the prognosis of the patients. However, none of these medicaments or other medicaments currently available, administered during reperfusion, is capable of limiting the size of a myocardial infarction.

An ischaemic state may also lead to impairment of the normal functioning of other organs, such as the kidneys (Zhao, Jing; Dong, et al. Guoji Bingli Kexue Yu Linchuang Zazhi (2007), 27(6), 539-544) or the brain (Zhu, Xia-Ling, Neuroscience Letters, 2009).

In recent years, various preclinical studies have demonstrated the capacity of sodium-proton exchanger (NHE) inhibitors in cardiac hypoperfusion conditions for protecting heart tissues that are at risk of being destroyed by the onset of an acute ischaemic event. Protection of the heart tissues with NHE inhibitors concerns any type of lesion or pathology caused by hypoperfusion, from cardiac arrhythmia to hypercontraction of the heart muscle and also temporary loss of function to necrosis of the heart tissues, associated with irreversible lesions.

The sodium-proton exchanger (NHE) plays an important role in acute ischaemic conditions. The mechanism of action of these NHE inhibitors consists in them reducing the increased influx of sodium ions into hypoperfused tissues by activating the NHE due to the intracellular acidification. This delays the excessive accumulation of sodium in the tissues. Insofar as the sodium and calcium ion transports are coupled with each other in heart tissues, this prevents potentially lethal overloading of the heart cells with calcium.

Moreover, European patent EP 1 250 328 discloses dihydro-1,3,5-triazine amine derivatives of the general formula (I) below:

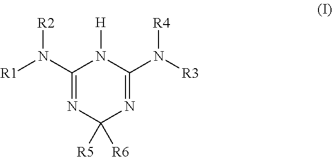

It has been demonstrated that these compounds have antidiabetic activity in an experimental model of non-insulin-dependent diabetes induced in rats using streptozotocin.

The present invention derives from the unexpected demonstration by the inventors that the combination of a sodium-proton exchanger inhibitor and a dihydro-1,3,5-triazine amine derivative of the formula (I) makes it possible to improve the treatment and/or prevention of diseases associated with ischaemia and/or reperfusion, especially cardiac and renal complications. More particularly, it has been demonstrated that the compound E 008 makes it possible to reduce the production of ROS by the complex I of the endothelial cell respiratory chain by inhibiting reverse electron flow. In addition, this compound makes it possible to prevent the loss of potential of the mitochondrial membrane and to reduce the opening of the mitochondrial transient permeability pore (TPP), especially during ischaemia and/or reperfusion events. These effects, combined with those of NHE inhibitors, such as cariporide, make it possible to improve the treatment or prevention of lesions, disorders or diseases associated with ischaemia-reperfusion.

The present invention thus relates to a combination of an NHE inhibitor and a compound of the general formula (I):

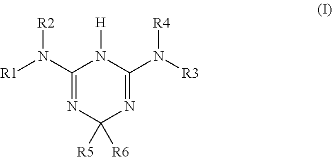

in which:
R1, R2, R3, and R4 are chosen independently from the groups:

H,

alkyl (C1-C20), which is optionally substituted by halogen, alkyl (C1-C5), alkoxy (C1-C5) or cycloalkyl (C3-C8), alkenyl (C2-C20), which is optionally substituted by halogen, alkyl (C1-C5) or alkoxy (C1-C5), or alkynyl (C2-C20), which is optionally substituted by halogen, alkyl (C1-C5) or alkoxy (C1-C5),

cycloalkyl (C3-C8), which is optionally substituted by alkyl (C1-C5) or alkoxy (C1-C5),

heterocycloalkyl (C3-C8), which bears one or more heteroatoms selected from N, O and S and is optionally substituted by alkyl (C1-C5) or alkoxy (C1-C5),

aryl (C6-C14) alkyl (C1-C20), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,

aryl (C6-C14), which is optionally substituted by amino, hydroxyl, thio, halogen, alkyl (C1-C5), alkoxy (C1-C5), alkylthio (C1-C5), alkylamino (C1-C5), aryloxy

(C6-C14), aryl(C6-C14)alkoxy(C1-C6), cyano, trifluoromethyl, carboxyl, carboxymethyl or carboxyethyl,

heteroaryl (C1-C13), which bears one or more heteroatoms selected from N, O and S and is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,

R1 and R2, on the one hand, and R3 and R4, on the other hand, possibly forming with the nitrogen atom an n-membered ring (n between 3 and 8) optionally containing one or more heteroatoms selected from N, O and S and possibly being substituted by one or more of the following groups: amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
R5 and R6 are chosen independently from the groups:

—H,

alkyl (C1-C20), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,

alkenyl (C2-C20), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,

alkynyl (C2-C20), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,

cycloalkyl (C3-C8), which is optionally substituted by amino, hydroxyl, thio, halogen, alkyl (C1-C5), alkoxy (C1-C5), alkylthio (C1-C5), alkylamino (C1-C5), aryl (C6-C14) oxy, aryl(C6-C14)alkoxy(C1-C6), cyano, trifluoromethyl, carboxyl, carboxymethyl or carboxyethyl,

heterocycloalkyl (C3-C8), which bears one or more heteroatoms selected from N, O and S and is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,

aryl (C6-C14), which is optionally substituted by amino, hydroxyl, thio, halogen, alkyl (C1-C5), alkoxy (C1-C5), alkylthio (C1-C5), alkylamino (C1-C5), aryl

(C6C14) oxy, aryl(C6-C14)alkoxy(C1-C6), cyano, trifluoromethyl, carboxyl, carboxymethyl or carboxyethyl,

heteroaryl (C1-C13), which bears one or more heteroatoms selected from N, O and S and is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,

aryl (C6-C14) alkyl (C1-C5), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,

R5 and R6 possibly forming, with the carbon atom to which they are attached, an m-membered ring (m between 3 and 8) optionally containing one or more heteroatoms selected from N, O and S and possibly substituted by amino, hydroxyl, thio, halogen, alkyl (C1-C5), alkoxy (C1-C5), alkylthio (C1-C5), alkylamino (C1-05), aryloxy (C6-C14), aryl(C6-C14)alkoxy(C1-C6), cyano, trifluoromethyl, carboxyl, carboxymethyl or carboxyethyl,

or possibly forming with the carbon atom a C10-C30 polycyclic residue, which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,

R5 and R6 together also possibly representing the group ═O or ═S,

the nitrogen atom of a heterocycloalkyl or heteroaryl group possibly being substituted by a group selected from: alkyl (C1-C5), cycloalkyl (C3-C8), aryl(C6-C14), aryl(C6-C14)alkyl(C1-C5) or acyl(C1-C6),

and also the tautomeric forms, enantiomers, diastereoisomers and epimers, and the pharmaceutically acceptable salts.

The present invention also relates to a pharmaceutical composition comprising a combination as defined above and a pharmaceutically acceptable vehicle.

The present invention also relates to a combination as defined above for its use in the treatment and/or prevention of a pathology associated with ischaemia and/or reperfusion.

The present invention also relates to the use of a combination as defined above for the preparation of a medicament for the prevention and/or treatment of a pathology associated with ischaemia and/or reperfusion.

The present invention also relates to a method for the prevention and/or treatment of a pathology associated with ischaemia and/or reperfusion in a patient, in which a prophylactically or therapeutically effective amount of a combination as defined above is administered to the patient.

### DETAILED DESCRIPTION OF THE INVENTION

The term “m-membered ring formed by R5 and R6” in particular means a saturated ring, such as a cyclohexyl, piperidyl or tetrahydropyranyl group.

The term “polycyclic group formed by R5 and R6” means an optionally substituted carbon-based polycyclic group and in particular a steroid residue.

One particular group of compounds of the formula (I) is that in which R5 is hydrogen.

Another particular group of compounds of the formula (I) is that in which R5 and R6 form, with the carbon atom to which they are attached, an m-membered ring (m between 3 and 8) optionally containing one or more heteroatoms selected from N, O and S and possibly substituted by one or more of the following groups:

alkyl (C1-C5), amino, hydroxyl, alkylamino(C1-C5), alkoxy(C1-C5), alkylthio(C1-C5), aryl (C6-C14), aryl(C6-C14)-alkoxy(C1-C5), or form with the carbon atom a C10-C30 polycyclic residue, which is optionally substituted by amino, hydroxyl, thio, halogen, alkyl (C1-C5), alkoxy (C1C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl.

Another particular group of compounds of the formula (I) is that in which R5 and R6 are chosen independently from the groups:

alkyl (C1-C20), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl.

The invention also relates to the tautomeric forms, the enantiomers, diastereoisomers and epimers, and the organic or mineral salts of the compounds of the general formula (I).

The compounds of the invention of the formula (I) as defined above containing a sufficiently acidic function or a sufficiently basic function, or both, may include the corresponding pharmaceutically acceptable salts of an organic or mineral acid or of an organic or mineral base.

In particular, the compounds of the general formula (I) contain basic nitrogen atoms that may be monosalified or disalified with organic or mineral acids.

Preferably, R6 is an alkyl (C1-C20) group, especially a methyl group.

Preferably, R1 and/or R2 represent(s) an alkyl (C1-C20) group, especially a methyl group.

Preferably, R3 and/or R4 represent(s) a hydrogen atom.

Preferably, R1 and R2 are a methyl group and R3 and R4 represent hydrogen.

Among the preferred compounds of the formula (I), particular mention may be made of compound E 008 of the formula (Ia):

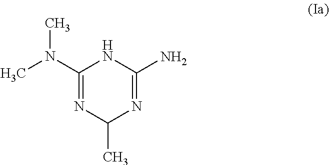

and also the tautomeric forms, enantiomers, diastereoisomers and epimers thereof and/or the pharmaceutically acceptable salts thereof.

The NHE inhibitor is preferably an NHE-1 inhibitor. By way of example, mention may be made, in particular, of amiloride or cariporide:

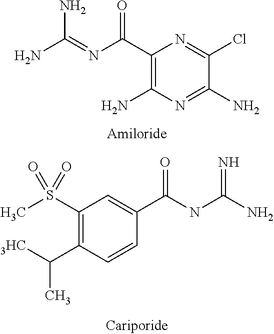

Other examples of NHE inhibitors that may be mentioned include sulfonyl- or sulfinylbenzoylguanidine derivatives of the formula (I) described as NHE inhibitors in patent application EP 758 644, especially compound A of the formula:

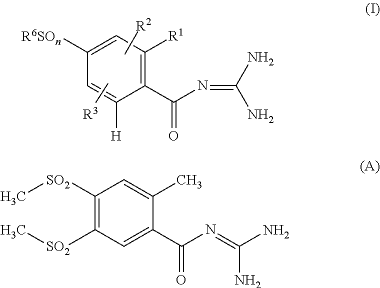

The NHE inhibitor is more preferably cariporide or a pharmaceutically acceptable salt thereof.

According to a second aspect, the invention relates to a pharmaceutical composition comprising a combination as defined above.

According to another aspect, the invention relates to a combination as defined above for use in the treatment and/or prevention of diseases associated with ischaemia-reperfusion, especially cardiac or renal complications. Examples of cardiac pathologies that may especially be mentioned include cardiac arrhythmia, myocardial infarction and cardiac hypertrophy, the latter generally inducing cardiac insufficiency.

In accordance with the present invention, the “alkyl” radicals represent saturated hydrocarbon-based radicals, in a straight or branched chain, of 1 to 20 carbon atoms and preferably 1 to 5 carbon atoms. If they are linear, particular mention may be made of methyl, ethyl, propyl, butyl, pentyl, hexyl, octyl, nonyl, decyl, dodecyl, hexadecyl and octadecyl radicals. If they are branched or substituted by one or more alkyl radicals, particular mention may be made of isopropyl, tert-butyl, 2-ethylhexyl, 2-methylbutyl, 2-methylpentyl, 1-methylpentyl and 3-methylheptyl radicals.

The “alkoxy” radicals according to the present invention are radicals of the formula —O-alkyl, the alkyl being as defined above.

The term “alkylthio” denotes a group alkyl-S—, the alkyl group being as defined above.

The term “alkylamino” denotes a group alkyl-NH—, the alkyl group being as defined above.

Among the halogen atoms, more particular mention may be made of fluorine, chlorine, bromine and iodine atoms.

The “alkenyl” radicals represent hydrocarbon-based radicals, in a straight or linear chain, and contain one or more ethylenic unsaturations. Among the alkenyl radicals, particular mention may be made of allyl or vinyl radicals.

The “alkynyl” radicals represent hydrocarbon-based radicals, in a straight or linear chain, and contain one or more acetylenic unsaturations. Among the alkynyl radicals, particular mention may be made of acetylene.

The “cycloalkyl” radical is a saturated or partially unsaturated, non-aromatic mono-, bi- or tricyclic hydrocarbon-based radical of 3 to 10 carbon atoms, especially, such as cyclopropyl, cyclopentyl, cyclohexyl or adamantyl, and also the corresponding rings containing one or more unsaturations.

The “heterocycloalkyl” radicals denote saturated or partially unsaturated, non-aromatic mono- or bicyclic systems of 3 to 8 carbon atoms containing one or more heteroatoms selected from N, O and S.

The term “aryl” denotes a mono- or bicyclic hydrocarbon-based aromatic system of 6 to 10 carbon atoms. Among the aryl radicals, particular mention may be made of phenyl or naphthyl radicals, more particularly substituted by at least one halogen atom.

The “arylalkyl” or “aralkyl” radicals are aryl-alkyl-radicals, the aryl and alkyl groups being as defined above. Among the arylalkyl radicals, particular mention may be made of the benzyl or phenethyl radical.

The term “aryloxy” denotes a group aryl-O—, the aryl group being as defined above.

The term “arylalkoxy” denotes an aryl-alkoxy-group, the aryl and alkoxy groups being as defined above.

The “heteroaryl” radicals denote mono- or bicyclic aromatic systems of 5 to 10 carbon atoms containing one or more heteroatoms selected from nitrogen, oxygen and sulfur. Among the heteroaryl radicals, mention may be made of pyrazinyl, thienyl, oxazolyl, furazanyl, pyrrolyl, 1,2,4-thiadiazolyl, naphthyridinyl, pyridazinyl, quinoxalinyl, phthalazinyl, imidazo[1,2-a]pyridine, imidazo[2,1-b]thiazolyl, cinnolinyl, triazinyl, benzofurazanyl, azaindolyl, benzimidazolyl, benzothienyl, thienopyridyl, thienopyrimidinyl, pyrrolopyridyl, imidazopyridyl, benzazaindole, 1,2,4-triazinyl, benzothiazolyl, furyl, imidazolyl, indolyl, triazolyl, tetrazolyl, indolizinyl, isoxazolyl, isoquinolinyl, isothiazolyl, oxadiazolyl, pyrazinyl, pyridazinyl, pyrazolyl, pyridyl, pyrimidinyl, purinyl, quinazolinyl, quinolinyl, isoquinolyl, 1,3,4-thiadiazolyl, thiazolyl, triazinyl, isothiazolyl and carbazolyl, and also the corresponding groups derived from their fusion or fusion with the phenyl nucleus.

The term “carboxyalkyl” denotes a HOOC-alkyl-group, the alkyl group being as defined above. Examples of carboxyalkyl groups that may be mentioned in particular include carboxymethyl and carboxyethyl.

The term “pharmaceutically acceptable salts” refers to relatively non-toxic mineral and organic acid-addition salts and base-addition salts of the compounds of the present invention. These salts can be prepared in situ during the final isolation and purification of the compounds. In particular, the acid-addition salts can be prepared by separately reacting the purified compound, in its purified form, Examples of acid-addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, sulfamates, malonates, salicylates, propionates, methylenebis(β-hydroxy)naphthoates, gentisic acid, isethionates, di-p-tolyltartrates, methanesulfonates, ethanesulfonates, benzenesulfonates, p-toluenesulfonates, cyclohexyl sulfamates and quinateslaurylsulfonate, and analogues (see, for example, S. M. Berge et al. “Pharmaceutical Salts” J. Pharm. Sci, 66: pp. 1-19 (1977)). The acid-addition salts can also be prepared by separately reacting the purified compound, in its acid form, with an organic or mineral base, and isolating the salt thus formed. The acid-addition salts include metal and amine salts. The suitable metal salts include the sodium, potassium, calcium, barium, zinc, magnesium and aluminium salts. The sodium and potassium salts are preferred. The suitable base mineral addition salts are prepared from metallic bases that include sodium hydride, sodium hydroxide, potassium hydroxide, calcium hydroxide, aluminium hydroxide, lithium hydroxide, magnesium hydroxide and zinc hydroxide. The suitable base amine-addition salts are prepared from amines that have sufficient alkalinity to form a stable salt, and preferably include amines that are often used in medicinal chemistry on account of their low toxicity and their acceptability for medical use: ammonia, ethylenediamine, N-methylglucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, for example lysine and arginine, and dicyclohexylamine, and analogues.

The compounds of the general formula (I) can be prepared by application or adaptation of any method known per se and/or within the scope of a person skilled in the art, especially those described by Larock in Comprehensive Organic Transformations, VCH Pub., 1989, or by application or adaptation of the processes described in EP 1 250 328.

The pharmaceutical compounds according to the invention may be in forms intended for parenteral, oral, rectal, permucous or percutaneous administration.

The pharmaceutical compositions including these compounds of the formula (I) will thus be in the form of injectable solutions or suspensions or multi-dose bottles, in the form of plain or coated tablets, dragees, wafer capsules, gel capsules, pills, cachets, powders, suppositories or rectal capsules, solutions or suspensions, for percutaneous use in a polar solvent, or for permucous use.

The excipients that are suitable for such administrations are cellulose or microcrystalline cellulose derivatives, alkaline-earth metal carbonates, magnesium phosphate, starches, modified starches, and lactose for the solid forms.

Cocoa butter or polyethylene glycol stearates are the preferred excipients for rectal use.

Water, aqueous solutions, physiological saline and isotonic solutions are the vehicles most conveniently used for parenteral use.

The dosage can vary within wide limits (0.5 mg to 1000 mg) as a function of the therapeutic indication and of the route of administration, and also of the age and weight of the individual.

The expression “lesions, disorders or diseases associated with ischaemia-reperfusion” denotes all lesions, disorders or diseases whose onset or maintenance finds its origin, at least partially, in ischaemia and/or reperfusion. It also denotes lesions, disorders or diseases that are the consequence of an ischaemic and/or reperfusion event.

The combination according to the invention is preferably useful for the treatment and/or prevention of cardiac complications, especially cardiac arrhythmia, myocardial infarction or cardiac insufficiency.

According to another preferred aspect, the combination is useful for the treatment of or preventing renal complications.

According to another preferred embodiment, the combination is useful for the treatment and/or prevention of cerebral complications, especially strokes.

### Example

### Evaluation of the Combined Protective Effect of E 008 and of an Na⁺/H⁺ Exchanger Inhibitor on Vascular Damage Induced by Ischaemia/Reperfusion

The object of this study is to evaluate the potential added effect of the combination of an Na⁺/H⁺ exchanger inhibitor and of the compound E 008, which is an inhibitor of the production of reactive oxygen species (ROS), on vascular damage induced by ischaemia/reperfusion, by measuring the effect of this combination on the loss of the mitochondrial membrane potential induced by ischaemia/reperfusion.

Materials and Methods

Cell Culture

The immortalized human dermal microvascular endothelial cell line HMEC-1 was used.

Treatment

The cells were treated according to the following scheme:
control group: no treatment, followed by ischaemia for 60 minutes and reperfusion for 120 minutes;
E 008 group: incubation with compound E 008 at a concentration of 10 micromolar to 10 millimolar for 30 minutes, followed by ischaemia for 60 minutes and reperfusion for 120 minutes;
metformin group: incubation with the compound metformin at a concentration of 10 micromolar to 10 millimolar for 30 minutes, followed by ischaemia for 60 minutes and reperfusion for 120 minutes;
cariporide group: incubation with cariporide at a concentration of 1 to 100 micromolar for 30 minutes, followed by ischaemia for 60 minutes and reperfusion for 120 minutes;
E 008+cariporide group: incubation with compound E 008 at a concentration of 10 micromolar to 10 millimolar and cariporide at a concentration of 1 to 100 micromolar for 30 minutes, followed by ischaemia for 60 minutes and reperfusion for 120 minutes;
metformin+cariporide group: incubation with the compound metformin at a concentration of 10 micromolar to 10 millimolar and cariporide at a concentration of 1 to 100 micromolar for 30 minutes, followed by ischaemia for 60 minutes and reperfusion for 120 minutes.

Measurement of the Mitochondrial Membrane Potential

In order to measure the mitochondrial potential, the cells were incubated, throughout the above treatment, with the fluorescent probe tetramethylrhodamine methyl ester (TMRM) at a concentration of 30 nM.

They were analysed by confocal microscopy before the treatment, after 60 minutes of ischaemia, after 90 minutes of reperfusion and after 120 minutes of reperfusion.

Results

In the control group and in the treated groups, the ischaemia period (60 minutes) did not modify the mitochondrial membrane potential.

On the other hand, the reperfusion for 90 minutes led to a substantial loss of the mitochondrial membrane potential in the control group. The membrane potential even disappeared after 120 minutes of reperfusion, which implies that the cells are dead.

In contrast, the cells treated with E 008 had their mitochondrial membrane potential protected against the damage induced by the reperfusion.

The combination of NHE and E 008 displays a protective synergistic effect towards ischaemia and reperfusion, this synergistic effect being translated by a significant decrease in the doses of E 008 used.

1. Combination of a sodium-proton exchanger (NHE) inhibitor and a compound of the formula (I): 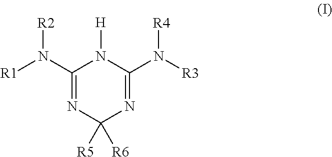
in which:
R1, R2, R3 and R4 are chosen independently from the groups:
H,
alkyl (C1-C20), which is optionally substituted by halogen, alkyl (C1-C5), alkoxy (C1-C5) or cycloalkyl (C3-C8), alkenyl (C2-C20), which is optionally substituted by halogen, alkyl (C1-C5) or alkoxy (C1-C5), or alkyne (C2-C20), which is optionally substituted by halogen, alkyl (C1-C5) or alkoxy (C1-C5),
cycloalkyl (C3-C8), which is optionally substituted by alkyl (C1-C5), alkoxy (C1-C5),
heterocycloalkyl (C3-C8), which bears one or more heteroatoms selected from N, O and S and is optionally substituted by alkyl (C1-C5) or alkoxy (C1-C5),
aryl (C6-C14) alkyl (C1-C20), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
aryl (C6-C14), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
heteroaryl (C1-C13), which bears one or more heteroatoms selected from N, O and S and is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
R1 and R2, on the one hand, and R3 and R4, on the other hand, possibly forming with the nitrogen atom an n-membered ring (n between 3 and 8) optionally containing one or more heteroatoms selected from N, O and S and possibly being substituted by one or more of the following groups: amino, hydroxyl, thio, halogen, alkyl (C1-C5), alkoxy (C1-C5), alkylthio (C1-05), alkylamino (C1-C5), aryloxy (C6-C14), aryl(C6-C14)alkoxy(C1-C6), cyano, trifluoromethyl, carboxyl, carboxymethyl or carboxyethyl,
R5 and R6 are chosen independently from the groups:
—H,
alkyl (C1-C20), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
alkenyl (C2-C20), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
alkynyl (C2-C20), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
cycloalkyl (C3-C8), which is optionally substituted by amino, hydroxyl, thio, halogen, alkyl (C1-C5), alkoxy (C1-C5), alkylthio (C1-C5), alkylamino (C1-C5), aryl (C6-C14) oxy, aryl(C6-C14)alkoxy(C1-C6), cyano, trifluoromethyl, carboxyl, carboxymethyl or carboxyethyl,
heterocycloalkyl (C3-C8), which bears one or more heteroatoms selected from N, O and S and is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
aryl (C6-C14), which is optionally substituted by amino, hydroxyl, thio, halogen, alkyl (C1-C5), alkoxy (C1-C5), alkylthio (C1-C5), alkylamino (C1-C5), aryl (C6C14) oxy, aryl(C6-C14)alkoxy(C1-C6), cyano, trifluoromethyl, carboxyl, carboxymethyl or carboxyethyl,
heteroaryl (C1-C13), which bears one or more heteroatoms selected from N, O and S and is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
aryl (C6-C14) alkyl (C1-C5), which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14) alkoxy(C1-C6), cyano, trifluoromethyl, carboxyl, carboxymethyl or carboxyethyl,
R5 and R6 possibly forming, with the carbon atom to which they are attached, an m-membered ring (m between 3 and 8) optionally containing one or more heteroatoms selected from N, O and S and possibly substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
or possibly forming with the carbon atom a C10-C30 polycyclic residue, which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
R5 and R6 together also possibly representing the group ═O or ═S,
the nitrogen atom of a heterocycloalkyl or heteroaryl group possibly being substituted by a group selected from: alkyl (C1-C5), cycloalkyl (C3-C8), aryl(C6-C14), aryl(C6-C14)alkyl(C1-C5) or acyl(C1-C6),
and also the tautomeric forms, enantiomers, diastereoisomers and epimers, and the pharmaceutically acceptable salts.

2. Combination according to claim 1, in which R5 is hydrogen.

3. Combination according to claim 1, in which R5 and R6:
form, with the carbon atom to which they are attached, an m-membered ring (m between 3 and 8), optionally containing one or more heteroatoms selected from N, O and S and possibly substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl,
or form with the carbon atom a C10-C30 polycyclic residue, which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl.

4. Combination according to claim 1, in which R5 is an alkyl (C2-C20) group substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl.

5. Combination according to claim 1, in which R5 and R6 are selected from alkyl (C1-C20) groups, which is optionally substituted by amino, hydroxyl, thio, halogen, alkyl (C1-C5), alkoxy (C1-C5), alkylthio (C1-C5), alkylamino (C1-C5), aryloxy (C6-C14), aryl(C6-C14)alkoxy(C1-C6), cyano, trifluoromethyl, carboxyl, carboxymethyl or carboxyethyl.

6. Combination according to claim 1, in which R6 is an alkyl (C1-C20) group.

7. Combination according to claim 6, in which R6 is a methyl group.

8. Combination according to claim 1, in which R1 and/or represents an alkyl (C1-C20) group.

9. Combination according to claim 1, in which R1 and R2 represent a methyl group.

10. Combination according to claim 1, in which R3 and/or R4 represent a hydrogen atom.

11. Combination according to claim 1, characterized in that
the compound of the formula (I) is the compound of the formula (Ia): 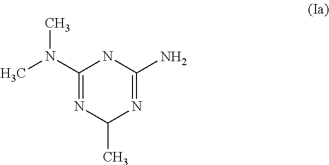
or the tautomeric forms, enantiomers, diastereoisomers and epimers thereof and/or pharmaceutically acceptable salts thereof.

12. Combination according to claim 1, in which the NHE inhibitor is cariporide or a pharmaceutically acceptable salt thereof.

13. Pharmaceutical composition comprising a combination according to claim 1.

14. Combination according to claim 1 for its use in the treatment and/or prevention of lesions, disorders or diseases associated with ischaemia and/or reperfusion.

15. Combination according to claim 15 for its use in the treatment and/or prevention of cardiac, renal or cerebral complications.

16. Combination according to claim 16 for its use in the treatment and/or prevention of cardiac arrhythmia, myocardial infarction or cardiac insufficiency.
